(19) United States
(12) Patent Application Publication
(10) Pub. No.: US 2005/0260290 A1
(43) Pub. Date: Nov. 24, 2005

(54) Botanical anti-inflammatory compositions and methods

(75) Inventors: Ily Raskin, Manalapan, NJ (US); Nebojsa Ilic, Highland Park, NJ (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: RUTGERS, THE STATE UNIVERSITY OF NEW JERSEY, New Brunswick, NJ (US)

(21) Appl. No.: 11/134,195

(22) Filed: May 19, 2005

Related U.S. Application Data
(60) Provisional application No. 60/572,798, filed on May 20, 2004.

(51) Int. Cl. A61K 31/192; A61K 35/78

(52) U.S. Cl. 424/756; 514/570

(57) ABSTRACT

The invention relates to anti-inflammatory compositions obtained from botanical sources. More specifically, the invention relates to anti-inflammatory compositions comprising an anti-inflammatory extract product of Aframomum melegueta, methods of making an anti-inflammatory composition, and methods of treating and preventing inflammatory responses.

### CROSS-REFERENCE TO RELATED APPLICATION

The benefit under 35 U.S.C. §119(e) of U.S. provisional patent application Ser. No. 60/572,798 filed May 20, 2004, the entire disclosure of which is incorporated herein by reference, is claimed.

### FIELD OF THE INVENTION

The invention relates to anti-inflammatory compositions obtained from botanical sources. More specifically, the invention relates to anti-inflammatory compositions comprising an anti-inflammatory extract product of Aframomum melegueta, methods of making an anti-inflammatory composition, and methods of treating and/or preventing inflammatory responses.

### BACKGROUND OF THE INVENTION

Inflammation is a localized response of a tissue to injury, e.g., an injury caused by an invading and/or infectious agent. An inflammatory response is frequently characterized by increased blood flow, increased temperature (e.g., fever), redness, swelling, and/or pain. Inflammatory responses are beneficial in that they can protect the body by isolating the site of injury, mobilizing effector cells thereto, and promoting healing.

Inflammatory responses can be disproportionate relative to the injury, however. For example, an inflammatory response can cause greater amounts of damage than the invading and/or infectious agent would have produced absent the inflammatory response. Additionally, inflammatory responses can be undesirably triggered in response to noninfectious agents in individuals having allergies and/or autoimmune diseases.

The arachidonic acid pathway constitutes one of the main cellular mechanisms for mediating inflammation. The arachidonic acid pathway includes the cyclooxygenase pathway and the 5-lipoxygenase pathway.

Prostaglandins are end products of the cyclooxygenase pathway. The enzymes involved in prostaglandin synthesis and the receptors to which prostaglandins bind are well-known pharmacological targets. For example, aspirin and other non-steroidal anti-inflammatory drugs (NSAIDs) reduce inflammation by inhibiting prostaglandin synthesis. Specifically, NSAIDs inhibit the activity of cyclooxygenase, an enzyme involved in prostaglandin synthesis.

Cyclooxygenase is present in the body in at least two isoforms, cyclooxygenase-1 (COX-1) and cyclooxygenase-2 (COX-2). COX-1 is expressed constitutively in most tissues and performs many ‘housekeeping’ functions, such as maintaining the protective lining of the stomach, regulating blood flow through kidneys, and promoting platelet aggregation, whereas COX-2 is an inducible isoform that is mainly produced in inflamed tissues [Dannenberg et al., Lancet Oncol., 2(9):544-51 (2001)].

NSAIDs inhibit both COX-1 and COX-2 activity. Thus, NSAIDs inhibit blood clotting by interfering with platelet aggregation (by inhibiting COX-1 activity) and by reducing inflammation (by inhibiting COX-2 activity).

In addition to its important role in inflammation, COX-2 is involved in regulating cellular proliferation, differentiation, and tumorigenesis. For example, increased levels of COX-2 expression in humans are found in various carcinomas, including colon, breast, lung, prostate, and pancreas carcinomas [Madaan et al., BJU International, 86(6):736-741 (2000)]. Additionally, several COX-2 inhibitor clinical trials suppressed carcinogenesis in human colon, prostate, and esophagus, suggesting that inhibiting COX-2 activity may be of therapeutic value for treating and/or preventing various cancers [Higashi et al., Int'l. J. Canc., 86(5):667-671 (2000)]. Similarly, NSAIDs have been demonstrated to be effective in treating and preventing prostate and colon cancers [Andrews et al, Cancer Chemother. Pharmacol., 50(4):227-284 (2002); Hixson et al., Cancer Epidemiol. Biomarkers Prev. 3(5):433-438 (1994)].

In light of these data, compounds that inhibit COX-2 or lower its expression are significant not only for the treatment of inflammatory responses, but also for human health and wellness in general [Huss et al., J. Nat. Prods., 65(11):1517-1521 (2002); Surh et al., Food Chem. Toxicol., 40(8):1091-1097].

### SUMMARY OF THE INVENTION

The invention provides anti-inflammatory compositions and methods for treating and preventing inflammatory responses.

One aspect of the invention provides an anti-inflammatory compositions derived from botanical sources. The disclosed botanical, anti-inflammatory compositions possess significant anti-inflammatory activity, and therefore are capable of inhibiting inflammation in an individual. Advantageously, the disclosed anti-inflammatory compositions are derived from easily cultivatable plants of the species Aframomum melegueta, also known as guinea pepper, alligator pepper, grains of paradise, and Amomum melegueta. In one embodiment of this aspect of the invention, the anti-inflammatory compositions include an anti-inflammatory extract product of an Aframomum melegueta plant, and at least one formulation agent selected from the group consisting of diluents, fillers, salts, binders and biologically acceptable carriers.

Another aspect according to the invention provides methods of making an anti-inflammatory composition comprising providing a plant material of an Aframomum melegueta plant, extracting the plant material with a fluid, and collecting the fluid, thereby obtaining an anti-inflammatory extract product of the Aframomum melegueta plant, and formulating the anti-inflammatory extract product with at least one formulation agent selected from the group consisting of diluents, fillers, salts, binders and biologically acceptable carriers.

In a further aspect, the invention provides methods for treating and/or preventing a condition involving an inflammatory response comprising administering a therapeutically effective amount of a composition comprising an anti-inflammatory extract product of an Aframomum melegueta plant to an individual. Methods and compositions in accordance with the invention have been used to measurably and safely reduce inflammation in an individual.

In yet a further aspect, the invention provides methods for treating and/or preventing cancer comprising administering a therapeutically effective amount of a composition comprising an anti-cancer extract product of an Aframomum melegueta plant to an individual.

### DETAILED DESCRIPTION OF THE INVENTION

The invention demonstrates that compositions comprising an anti-inflammatory extract product of Aframomum melegueta possess significant anti-inflammatory activity. The anti-inflammatory activity of the compositions is generally attributed to the ability of the compositions to inhibit cyclooxygenase. The anti-inflammatory compositions in accordance with the invention comprise compounds derived from natural sources that effectively suppress, inhibit, reduce, or otherwise curtail inflammation (i.e., effectively treat inflammation) in an individual, for example, because they inhibit COX-2 activity. Thus, the anti-inflammatory compositions can also be administered to an individual to treat or prevent a condition involving an inflammatory response, for example, because they inhibit COX-2 activity. Additionally, the anti-inflammatory compositions may be formulated as pharmaceutical compositions (e.g., an ethical drug), nutraceutical compositions (e.g., a dietary supplement), cosmeceuticals (e.g., a cosmetic product having biologically active ingredients), or as a food or beverage additive as defined by the U.S. Food and Drug Administration.

As used herein, the term “extract product” refers to any compound, any agent and/or mixtures thereof, that is obtained, isolated, and/or derived from an extract of a plant material. The term “plant material” refers to any plant material including, but not limited to, leaves, stems, flowers, fruits, seeds, roots, and combinations thereof.

The anti-inflammatory compositions in accordance with the invention advantageously comprise an anti-inflammatory extract product of Aframomum melegueta, an easily cultivatable, edible, agricultural crop that has been extensively used as a food spice. Afromomum melegueta, also known as guinea pepper, alligator pepper, grains of paradise, and Amomum melegueta, Roskoe, is a plant of West African origin.

As used herein, “anti-inflammatory” refers to a statistically significant and detectable or measurable reduction in inflammation (e.g., induced inflammation) that is observed in individuals that have been treated with an anti-inflammatory composition in accordance with the invention, relative to individuals that have not been similarly treated. Typically, inflammation is induced in an individual, and the individual is then treated with an anti-inflammatory composition of the invention. Inflammation is then measured at a time period (e.g., after about 1 hour or more) subsequent to induction of inflammation and treatment (if any) with the anti-inflammatory compositions of the invention.

As used herein, the term “inflammatory response” is characterized by at least one, and preferably all, of the following symptoms: redness, heat, swelling and pain (i.e., inflammation); typically, inflammation involves tissue injury or destruction. An inflammatory response is usually a localized, protective response elicited by injury or destruction of tissues, which serves to destroy, dilute or wall off (sequester) both the injurious agent and the injured tissue. Inflammatory responses are notably associated with the influx of leukocytes and/or leukocyte (e.g., neutrophil) chemotaxis. Inflammatory responses may result from infection with pathogenic organisms and viruses, noninfectious states or processes such as trauma or reperfusion following myocardial infarction or stroke, immune responses to foreign antigens, and autoimmune diseases. Inflammatory responses amenable to treatment with the methods and compounds according to the invention encompass conditions associated with reactions of the specific immune defense system as well as conditions associated with reactions of the non-specific immune defense system.

As used herein, the term “inflammation” is a localized tissue response characterized by, but not limited to, increased blood flow, increased temperature (including, but not limited to, fever), redness, swelling, and/or pain.

The therapeutic, anti-inflammatory compositions and methods of the invention include methods for the amelioration of disorders associated with inflammatory cell activation. “Inflammatory cell activation” refers to the induction by a stimulus (including, but not limited to, cytokines, antigens or auto-antibodies) of a proliferative cellular response, the production of soluble mediators (including, but not limited to, cytokines, oxygen radicals, enzymes, prostanoids, or vasoactive amines), or cell surface expression of new or increased numbers of mediators (including, but not limited to, major histocompatability antigens or cell adhesion molecules) in inflammatory cells (including, but not limited to, monocytes, macrophages, T lymphocytes, B lymphocytes, granulocytes (polymorphonuclear leukocytes including neutrophils, basophils, and eosinophils), mast cells, dendritic cells, Langerhans cells, and endothelial cells). It will be appreciated by persons skilled in the art that the activation of one or a combination of these phenotypes in these cells can contribute to the initiation, perpetuation, or exacerbation of an inflammatory response.

In one aspect, the invention provides methods of inhibiting an inflammatory response comprising administering a therapeutically effective amount of a composition comprising an anti-inflammatory extract product of a plant material of an Aframomum melegueta plant to an individual. It is contemplated that inhibition of inflammation associated with a variety of disorders will beneficially affect the development, progression, and/or duration of such disorders.

Thus, in one embodiment, the term “therapeutically effective amount” refers to an amount of a composition comprising an anti-inflammatory extract product that is sufficient to reduce, decrease, and/or inhibit an inflammatory response in an individual. In an alternative embodiment, the term “therapeutically effective amount” refers to an amount of a composition comprising an anti-inflammatory extract product that is sufficient to alleviate, ameliorate, prevent, and/or eliminate at least one symptom known to be associated with a condition and/or the pathology of a condition involving an inflammatory response. Generally, the condition is selected from the group consisting of autoimmune diseases, arthritic diseases, dermatitis, and allergic diseases.

“Autoimmune disease” as used herein refers to any of a group of disorders in which tissue injury is associated with humoral or cell-mediated responses to the body's own constituents. “Arthritic disease” as used herein refers to any disease that is characterized by inflammatory lesions of the joints. “Dermatitis” as used herein refers to any of a large family of diseases of the skin that are characterized by inflammation of the skin. “Allergic disease” as used herein refers to any symptoms, tissue damage, or loss of tissue function resulting from allergy.

Thus, in various embodiments, specific conditions and/or disease states involving inflammation that may be treated with the methods and compounds of the invention include, but are not limited to, autoimmune diseases such as connective tissue disease, autoimmune pulmonary inflammation, Guillain-Barre syndrome, some forms of diabetes such as insulin-dependent diabetes mellitis, myasthenia gravis, autoimmune inflammatory eye disease, systemic lupus erythematosus (SLE), lupus nephritis, autoimmune thyroiditis, multiple sclerosis, and Reynaud's syndrome; arthritic diseases such as rheumatoid arthritis (RA), osteoarthritis, gouty arthritis, spondylitis, and reactive arthritis; Behcet's syndrome; inflammatory dermatitis such as contact dermatitis, atopic dermatitis, psoriasis, and urticaria.

The methods and compositions of the invention may also be useful in the treatment of allergic diseases, reactions, and conditions (e.g., anaphylaxis, serum sickness, drug reactions, food allergies, insect venom allergies, mastocytosis, allergic rhinitis, hypersensitivity pneumonitis, urticaria, angioedema, eczema, atopic dermatitis, allergic contact dermatitis, erythema multiforme, Stevens-Johnson syndrome, allergic conjunctivitis, atopic keratoconjunctivitis, venereal keratoconjunctivitis, giant papillary conjunctivitis and contact allergies), such as asthma (particularly allergic asthma) or other respiratory problems.

Moreover, in another embodiment of the invention, the compositions can also be administered to an individual to treat and/or prevent cancer because they inhibit COX-2 activity. Thus, the invention provides methods for treating or preventing cancer comprising administering a therapeutically effective amount of a composition comprising an anti-inflammatory extract product of a plant material of an Aframomum melegueta plant to an individual.

Cancer, as used herein, generally includes solid tumors, hematological cancers (including, but not limited to, multiple myeloma and leukemias), and lymphomas. According to this embodiment, the term “therapeutically effective amount” refers to an amount of a composition comprising an anti-cancer extract product that is sufficient to reduce, decrease, and/or inhibit-a cancerous growth in an individual. In an alternative embodiment, the term “therapeutically effective amount” refers to an amount of a composition comprising an anti-cancer extract product that is sufficient to alleviate, ameliorate, prevent, and/or eliminate the symptoms and/or the pathology of a cancer.

As demonstrated herein, extract products obtained from Aframomum melegueta plants include compounds and/or agents having anti-inflammatory activity (“anti-inflammatory compounds”). The anti-inflammatory activity of Aframomum melegueta extract products (and thus, the disclosed anti-inflammatory compositions) is generally attributed to the presence of one or more compounds in accordance with the following formula (I): 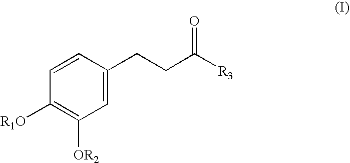
wherein R₁ is hydrogen or a C₁-C₄ moiety;
R₂ is hydrogen or a C₁-C₄ moiety; and,
R₃ is a C₁-C₁₀ moiety.

As used herein, the term “C₁-C₄ moiety” includes from one to four carbon atoms. Carbon-carbon bonds may be saturated or unsaturated. Non-carbon atoms may be bound to the carbon backbone, either directly or indirectly. Typical examples include alkyl, alkylene, heteroalkyl, and alkenyl groups as defined herein.

As used herein, the term “C₁-C₁₀ moiety” includes from one to ten carbon atoms. Carbon-carbon bonds may be saturated or unsaturated. Non-carbon atoms may be bound to the carbon backbone, either directly or indirectly. Typical examples include alkyl, alkylene, heteroalkyl, and alkenyl groups as defined herein.

“Alkyl” as used herein includes straight chain and branched hydrocarbon groups. “Alkylene” as used herein refers to alkyl groups (as defined) further including one or more substituents. Additionally, “heteroalkyl” as used herein refers to alkyl groups further containing a heteroatom such as O, P, S, or N. “Alkenyl” as used herein refers to alkyl groups further containing one or more carbon-carbon double bonds.

Most typically, R₁ is hydrogen and R₂ is methyl. In one embodiment where R₁ is hydrogen and R₂ is methyl, R₃ is 2-hydroxy heptane as shown below in formula II: 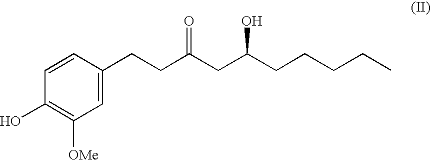
The compound depicted in formula II is (5S)-5-hydroxy-1-(4-hydroxy-3-methoxyphenyl)-3-decanone, also known as 6-gingerol. Although the S enantiomer is shown above, both racemic mixtures and isolated, optically active enantiomers (R, S) are contemplated for use in the compositions and methods of the invention when molecules have a stereocenter. Additionally, other gingerol structures (e.g., 8-gingerol) may be present in, or prepared from (i.e., synthetically derived from), the extract products in accordance with the invention.

In another embodiment where R₁ is hydrogen and R₂ is methyl, R₃ is heptane as shown in formula III: 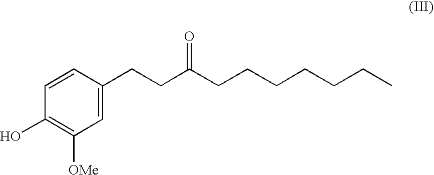
The compound depicted in formula III is 1-(4-hydroxy-3-methoxyphenyl)-3-decanone, also known as 6-paradol. Additionally, other paradol structures (e.g., 8-paradol) may be present in, or prepared from, the extract products in accordance with the invention.

In yet another embodiment where R₁ is hydrogen and R₂ is methyl, R₃ is hepta-1-ene as shown in formula IV: 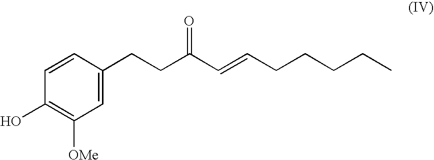
The compound depicted in formula IV is 1-(4-hydroxy-3-methoxyphenyl)-deca-4-ene-3-one, also known as 6-shogaol. Additionally, other shogaol structures may be present in, or prepared from, the extract products in accordance with the invention.

The disclosed anti-inflammatory compositions typically contain a mixture of anti-inflammatory compounds in accordance with formulas (I), (II), (III), and/or (IV). Accordingly, the invention contemplates mixtures, which may exhibit additive, or preferably synergistic, effects. With reference to the data described in Example 6, it can be seen that an anti-inflammatory extract product in accordance with the invention (that comprises a mixture of compounds) has a COX-2 inhibitory activity greater than any of the individual compounds present in the mixture. Thus, a mixture of compounds in accordance with formula I, for example, a mixture of compounds in accordance with formulas II, III, and IV, is a potentiating mixture.

Aframomum melegueta plants are grown and harvested using well-known methods. For example, the plants may be grown in an agricultural field. More preferably, the plants are grown in environmentally controlled hydroponic greenhouses using standard hydroponic methods. Hydroponic methods facilitate the reproducible optimization of plant growing conditions, and the optimization of anti-inflammatory compound content. Hydroponic methods also facilitate harvesting of the plants. Additionally, controlled growth conditions are advantageous in that they facilitate the standardization of any final product.

The conditions under which the plants are grown may also affect the anti-inflammatory compound content. In particular, plants subjected to stress conditions, such as heat stress, dehydration, and/or exposure to chemical elicitors, are expected to have a higher anti-inflammatory compound content than plants not subjected to such conditions. Any conventionally known chemical elicitor can be used during cultivation of the Aframomum melegueta plants, in accordance with known application schedules.

As previously described, the anti-inflammatory compounds are typically isolated by extracting plant material of an Aframomum melegueta plant. Any plant material, including leaves, stems, flowers, fruits, roots, and combinations thereof, can be extracted. In one embodiment, the above-ground plant parts are extracted. In a further embodiment, the seeds are used (by themselves).

One exemplary extraction method for obtaining high yields of anti-inflammatory compounds from Aframomum melegueta plants in accordance with the invention comprises the following steps: (1) providing fresh or fresh-frozen plant material; (2) disrupting the plant material; (3) extracting the plant material in a solution containing a sufficient amount of fluid; and (4) collecting the fluid to obtain an extract product. The anti-inflammatory extract product may be further processed by: (5) removing solid matter from the extract; (6) removing fluid components; (7) resuspending the resulting residue in an aqueous solution; and (8) after removing any water insoluble material, repeating step (6) to form a more purified form of an extract product. In various embodiments, the plant material can be disrupted by macerating, grinding, or otherwise disrupting the plant material.

In a preferred embodiment, fresh plant tissue is quick-frozen in liquid nitrogen, then ground or otherwise macerated (e.g., using a Polytron or a Waring blender) in fluid. After solids are removed from the extract, e.g., by filtration, centrifugation, or any method known in the art, the anti-inflammatory compound content of the extract can optionally be measured by any known method, including spectrometric methods.

Fluids for use in the extraction methods of the invention may be solvents. Suitable fluids include, but are not limited to, water, alcohols, alkanes, halocarbons, ethers, aromatic solvents, ketones, aqueous solutions, and super critical fluids. In one embodiment, ethanol is a preferred alcohol for practice of the invention. A benefit of incorporating an ethanolic fluid in the final extraction step is that an ethanolic fluid is compatible with an ingestible product, and therefore is suitable for incorporation into a pill, capsule, tablet, and other ingestible forms known in the art.

As previously indicated, the anti-inflammatory compositions may be formulated as pharmaceutical compositions (e.g., an ethical drug), nutraceutical compositions (e.g., a dietary supplement), compositions for topical administration including, but not limited to, cosmeceuticals (e.g., a cosmetic product having biologically active ingredients), or as a food or beverage additive as defined by the U.S. Food and Drug Administration. In one embodiment, the anti-inflammatory compositions include at least one formulation agent selected from the group consisting of diluents, fillers, salts, binders and biologically acceptable carriers.

Anti-inflammatory compositions comprising an anti-inflammatory extract product can be formulated as gels, ointments, lotions, creams, sprays, drops, suppositories, sprays, transdermal patches, or otherwise formulated for topical administration. Anti-inflammatory compositions formulated for topical administration typically include biologically acceptable carriers (i.e., a carrier that does not interfere with the anti-inflammatory activity of the Aframomum melegueta-derived anti-inflammatory extract product). Suitable biologically acceptable carriers are well known in the art and include, but are not limited to, oils and esters. Specific examples include mineral oil, glyceryl stearate, stearic acid, glycerin, silicone 1401, and propylene glycol.

Additionally, cosmeceutical compositions of the present invention can include a wide range of additional components. The CTFA Cosmetic Ingredient Handbook, Second Edition, 1992, which is incorporated by reference herein in its entirety, describes a wide variety of cosmetic and pharmaceutical ingredients commonly used in the skin care industry, which are suitable for use in the compositions of the present invention. Nonlimiting examples of functional classes of ingredients are described at page 537 of this document. Examples of these functional classes include: absorbents, abrasives, anti-acne agents, anticaking agents, antifoaming agents, antimicrobial agents, antioxidants, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, plasticizers, preservatives, propellants, reducing agents, skin bleaching agents, skin-conditioning agents (emollient, humectants, miscellaneous, and occlusive), skin protectants, solvents, foam boosters, hydrotropes, solubilizing agents, suspending agents (nonsurfactant), sunscreen agents, ultraviolet light absorbers, and viscosity increasing agents (aqueous and nonaqueous). Examples of other functional classes of materials useful herein that are well known to one of ordinary skill in the art include emulsifiers, sequestrants, skin sensates, and the like.

Anti-inflammatory compositions comprising an anti-inflammatory extract product can be tabletted, encapsulated, or otherwise formulated for oral administration (e.g., in a gum or candy). Compositions formulated for oral administration typically include one or more suitable diluents, fillers, salts, disintegrants, binders, lubricants, glidants, wetting agents, controlled release matrices, colorants, flavorings, carriers, excipients, buffers, stabilizers, solubilizers, commercial adjuvants, and/or other additives known in the art.

Any pharmaceutically acceptable (i.e., sterile and acceptably non-toxic, as known in the art) liquid, semisolid, or solid diluent that serves as a pharmaceutical vehicle, excipient, or medium can be used. Exemplary diluents include, but are not limited to, polyoxyethylene sorbitan monolaurate, magnesium stearate, calcium phosphate, mineral oil, cocoa butter, oil of theobroma, methyl- and propylhydroxybenzoate, talc, alginates, carbohydrates, especially mannitol, α-lactose, anhydrous lactose, cellulose, sucrose, dextrose, sorbitol, modified dextrans, gum acacia, and starch. Such additives may influence the physical state, stability, rate of in vivo release, and rate of in vivo clearance of the present anti-inflammatory compounds.

Pharmaceutically acceptable fillers can include, for example, lactose, microcrystalline cellulose, dicalcium phosphate, tricalcium phosphate, calcium sulfate, dextrose, mannitol, sucrose, and others known in the art. Salts, including calcium triphosphate, magnesium carbonate, and sodium chloride, may also be used as fillers in the pharmaceutical compositions.

Binders may be used to hold the composition comprising the anti-inflammatory extract product together to form a hard tablet. Exemplary binders include materials from natural products such as acacia, tragacanth, starch and gelatin. Other suitable binders include methyl cellulose (MC), ethyl cellulose (EC), and carboxymethyl cellulose (CMC).

As set forth in Examples 3-5, methods in accordance with the invention have been used to measurably and safely reduce inflammation. The methods for reducing inflammation include administering to an individual a therapeutically effective amount of a composition comprising an anti-inflammatory extract product of a plant material of an Aframomum melegueta plant.

The compositions (and thus the methods) of the invention can be used alone or in conjunction with other therapies including, for example, administration of other therapeutic agents (including other anti-inflammatory compositions or formulations). The methods in accordance with the invention contemplate administration of composition comprising an anti-inflammatory extract product-whether or not symptoms are manifest, i.e., prophylactic administration is contemplated. Preferred dosages of anti-inflammatory therapeutics such as naproxen and ibuprofen are known in the art for a variety of therapeutic and prophylactic purposes; in like manner, appropriate dosages of the anti-inflammatory compositions in accordance with the invention may be easily determined by standard methods.

It will be appreciated that the treatment methods of the invention are useful in the fields of human medicine and veterinary medicine. Thus, the subject or individual to be treated may be a mammal, preferably human, or other animals. For veterinary purposes, subjects include, for example, farm animals such as cows, sheep, pigs, horses, and goats; companion animals such as dogs and cats; exotic and/or zoo animals; laboratory animals including mice, rats, rabbits, guinea pigs, and hamsters; and poultry such as chickens, turkeys, ducks, and geese. A related aspect of the invention provides a use for the biologically active compositions or compounds in preparing medicaments for the treatment or prevention of the disorders disclosed herein.

In the methods according to the invention, the anti-inflammatory compositions may be administered by any known route of administration. For example, a composition comprising an anti-inflammatory extract product of an Aframomum melegueta plant can be formulated for injection, or for oral, nasal, transdermal or other forms of administration. Typically, the anti-inflammatory compositions are formulated for oral or topical administration. In some embodiments, the anti-inflammatory compositions are prepared using a non-toxic alcohol or an aqueous solution.

A typical treatment course may comprise administration of multiple doses on a daily basis of a composition comprising an amount of an anti-inflammatory extract product effective to inhibit an inflammatory response in an individual. Such a treatment course may be continued for significant periods of time, for example, three doses per day over three months or even indefinitely. In one embodiment, a presently preferred dosing schedule is one dose per day. The treatment may be continued on an as-needed basis.

Additionally, the compositions may be administered to an individual at any time of day. Typically, the compositions are administered at least one hour before consumption of food is anticipated.

Of course, the foregoing are only exemplary treatment schedules, and other schedules are contemplated. In each case, the suitability of such schedules and the aforementioned modes of administration are determined by those of skill in the art, using routine procedures. For example, those of skill in the art will be able to take the information disclosed in this specification and optimize treatment regimes for human subjects based on clinical trials performed in accordance with the specification.

### EXAMPLES

The following examples are provided to describe the invention in greater detail, and are intended to illustrate, not to limit, the appended claims. Example 1 provides an exemplary method for preparing an anti-inflammatory extract product of Aframomum melegueta. Example 2 provides in vitro evidence that anti-inflammatory compositions each comprising an anti-inflammatory extract product of Aframomum melegueta inhibit inflammation. Examples 3-5 provide in vivo evidence that anti-inflammatory compositions, each comprising an anti-inflammatory extract product of Aframomum melegueta, inhibit inflammation. Example 6 describes exemplary methods for purifying and preparing an anti-inflammatory extract product, and provides data concerning the activity of individual compounds isolated therefrom.

### Example 1

### Method of Extracting an A. Melegueta Plant Material

An anti-inflammatory extract product was prepared by extracting dry, ground seeds of Aframomum melegueta (grains of paradise) in 95 vol. % ethanol in about one part weight (milligrams of ground seeds) to about 10 parts volume (milliliters of solvent) ratio for 24 hours at room temperature. During the extraction process, a platform shaker was used to continuously agitate the ethanolic fluid to facilitate complete extraction of the ground seeds. After 24 hours, the fluid was filtered, and removed by rotary evaporation to provide an Aframomum melegueta extract product.

### Example 2

### COX-2 Inhibitory Activity of an A. Melegueta Extract Product

The Aframomum melegueta-derived anti-inflammatory extract product of Example 1 was tested in vitro for COX-2 inhibition.

The anti-inflammatory extract product of Example 1 was suspended in 95 vol. % ethanol at a concentration of 1 milligram (mg) extract product per milliliter (mL) ethanol to form an A. melegueta solution. The activity of the solution was tested with a colorimetric COX (ovine) inhibitor screening assay (catalogue no. 760111, Cayman Chemical, Ann Arbor, Mich.). The screening assay measures the peroxidase activity of cyclooxygenases by monitoring the appearance of oxidized N,N,N′N′-tetramethyl-p-phenylenediamine (TMPD) at 590 nm as previously described [Kulmacz, Prostaglandins, 25:531-540 (1983)]. A (relatively) higher TMPD concentration corresponds to a greater absorbance at 590 nm, a greater peroxidase activity, and a greater COX-2 activity (corresponding to the degree of inhibition of COX-2 activity).

Three milliliters (mL) of assay buffer concentrate (as provided) was diluted with 27 mL HPLC-grade water to provide a final assay buffer (0.1M Tris-HCl, pH 8). This buffer assay solution was used to prepare heme and COX-2 enzyme assay solutions. 88 microliters (μl) heme in dimethyl sulfoxide (DMSO) (as provided) was diluted with 1.912 milliliters (mL) buffer solution to provide a heme assay solution. 200 μl of COX-2 enzyme (as provided) was diluted with 760 μl of final buffer assay solution to provide a COX-2 assay solution. 100 μl of 0.1M KOH and 1.8 mL HPLC-grade water were added to 100 μl arachidonic acid in ethanol (as provided) to form a 1.1 mM arachidonic acid assay solution. The calorimetric substrate solution (TMPD) was used as provided.

160 μl of buffer assay solution and 10 μl of heme assay solution were added to three wells of a 96-well plate which were used as background wells. 150 μl of final assay buffer solution, 10 μl of heme assay solution, and 10 μl of COX-2 assay solution were added to three wells of a 96-well plate that were used as negative control wells. 150 μl of final assay buffer solution, 10 μl of heme assay solution, and 10 μl of COX-2 assay solution were added to three wells of a 96-well plate which were used as inhibitor wells (i.e., wells for testing the inhibitory activity of the A. melegueta solution). 150 μl of final assay buffer solution, 10 μl of heme assay solution, and 10 μl of COX-2 assay solution were added to three wells of a 96-well plate which were used as positive control wells.

10 μl of 95 vol. % ethanol was then added to the background and negative control wells. 10 μl of the A. melegueta solution was added to the inhibitor wells. 10 μl of an ethanolic solution containing Vioxx® (Merck, Inc.) at a concentration of 1 mg/mL was added to the positive control wells. The reagents were carefully mixed by shaking the plate, followed by incubation at 25° C. for five minutes. 20 μl of the colorimetric substrate solution was added to each of the wells. 20 μl of the 1.1 mM arachidonic acid assay solution was added to each well. The plate was again shaken and incubated for an additional 5 minutes at 25° C. The absorbance for each well was measured at 590 nm using a plate reader.

The inhibitory activity was calculated from these absorbance readings. The A. melegueta solution demonstrated high inhibitory activity against COX-2 enzyme relative to the same concentration of the commercial drug Vioxx® (Merck, Inc.), which was used as the postive control. The level of inhibition at concentrations of 1 mg/mL was 92% for the A. melegueta solution compared to 90% for Vioxx®. In addition to the significant COX-2 inhibitory activity, there are some data that suggest that the extract products also beneficially inhibit COX-1 enzyme.

Additionally, the dose-response curve was established for five concentrations of the extract (0.01, 0.05, 0.1, 0.5 and 1 mg/mL). Based on the dose response relationship, the IC₅₀ (the concentration of anti-inflammatory composition concentration that shows 50% enzyme activity as compared to the activity in the absence of any inhibitor) of the extract was determined to be 0.2 mg/mL.

This example demonstrates that Aframomum melegueta extract products effectively inhibit COX-2 enzyme activity.

### Example 3

### Demonstration of Anti-Inflammatory Activity of A. Melegueta Extract Product in Hind Paw Edema Model

The anti-inflammatory extract product of Example 1 was tested in vivo in a rat hind paw edema assay to evaluate the in vivo anti-inflammatory activity of the Aframomum melegueta extract products relative to a positive control. Hind paw edema models have been extensively described [see, for example, Winyard et al. (eds.), Inflammation Protocols, pp. 115-122 (2003)] and have been accepted as in vivo models of human inflammation.

The anti-inflammatory extract product of Example 1 was administered orally at two different doses (1000 mg/kg and 500 mg/kg) to two different groups of three Long-Evans-derived (male or female) overnight fasted rats weighing 150±20 g one hour before right hind paw injection of carrageenan (0.1 ml of a suspension of 1 wt. % carrageenan in 95 vol. % ethanol, intraplantar). Aspirin was administered to another group of three Long-Evans rats at 150 mg/kg dose as a positive control reference compound. The severity of the hind paw edema (i.e., the hind paw volume) was recorded 3 hours after carrageenan administration using a plethysmometer (Ugo Basile, Italy). The hind paw volume measurements correspond to the amount of induced swelling, and therefore a reduction in the swelling corresponds to a reduction in inflammation. The anti-inflammatory activity of the Aframomum melegueta extract products can be expressed as a percentage of edema reduction (relative to the negative control (i.e., full edema)).

At the dose of 1000 mg/kg, the anti-inflammatory extract product demonstrated significant anti-inflammatory effect by inhibiting the edema by about 49%. At the lower dose of 500 mg/kg, the extract inhibited the edema by about 11%. The positive control, aspirin at 150 mg/kg, reduced the edema by about 43%.

This example demonstrates that Aframomum melegueta extract products can be used to effectively and safely reduce inflammation in an individual.

### Example 4

### Demonstration of Anti-Inflammatory Activity of A. Melegueta Extract Product in Hind Paw Edema Model

The anti-inflammatory extract product of Example 1 was again tested in vivo in a rat hind paw edema assay to evaluate the in vivo anti-inflammatory activity of the Aframomum melegueta extract products. Two studies were conducted.

In both studies, the anti-inflammatory extract product of Example 1 was administered orally to groups of five, similarly-sized, Long-Evans-derived (male or female) rats. The anti-inflammatory extract product was administered to the groups at different dose protocols. Comparisons can be made relative to the negative and positive control animal groups.

Groups 2 and 4 of the first and second study, and Groups 2 and 5 of the second study, received a single dose of the extract product one hour after hind paw injection of carrageenan (0.1 ml of a suspension of 1 wt. % carrageenan in 95 vol. % ethanol, intraplantar). Group 3 of both studies received a first dose of the extract product one hour before, and a second dose of the extract product one hour after, hind paw injection of carrageenan. Group 1 of both studies did not receive any anti-inflammatory compounds, and thus represent negative control groups. Group 4 of the second study received a dose of the extract product one hour before administration of carrageenan. Group 5 of the first study and Group 6 of the second study received indomethacin, a commercially available nonsteroidal anti-inflammatory drug, one hour after hind paw injection of carrageenan. Indomethacin was administered as a positive control reference compound, and Groups 5 and 6 (of the first and second studies, respectively) represent positive control groups.

The severity of the hind paw edema (i.e., hind paw volume) was recorded at 3 hours after, 5 hours after, and 24 hours after carrageenan administration using a plethysmometer (Ugo Basile, Italy). The hind paw volume measurements after carrageenan administration correspond to the amount of induced swelling, and a reduction in the percentage of increase in hind paw volume relative to the percentage of increase observed in hind paw volume for the negative control group corresponds to a reduction in inflammation. For example, the hind paw volume of the negative control group of the first study (Group 1) increased by about 45% whereas the hind paw volume of Group 2 (which received an Aframomum melegueta extract product) increased by only 25%. Thus, Group 2 experienced about 44% less inflammation than the negative control group.

The results of the studies are shown in Tables 1 and 2, below, and demonstrate significant anti-inflammatory activity for the Aframomum melegueta extract products. Importantly, each group of animals that received an Aframomum melegueta extract product exhibited a reduction in the percentage of induced inflammation relative to the induced inflammation observed for the negative control groups. Additionally, Groups 3 of both studies received a dual dose of Aframomum melegueta extract products (i.e., before and after carrageenan administration), and experienced less inflammation than Group 5 of the first study and Group 6 of the second study, respectively, which received the positive control compound indomethacin.

This example also demonstrates that Aframomum melegueta extract products can be used to reduce inflammation in an individual.

TABLE 1

| | | Initial paw | Increase in rat hind paw volume after carrageenan | | | |
|---|---|---|---|---|---|---|
| | | volume (ml) ± | administration as a function of time (h) | | | |
| Group | Treatment | standard deviation | 3 h | 5 h | 24 h | 48 h |
| 1. | Carrageenan | 1.24 ± 0.08 | 1.80 ± 0.07 | 1.62 ± 0.13 | 1.48 ± 0.08 | 1.24 ± 0.05 |
| | (negative control) | | 45% | 31% | 19% | 0% |
| 2. | Carrageenan and 500 mg/kg | 1.1 ± 0.07 | 1.38 ± 0.24 | 1.28 ± 0.008 | 1.30 ± 0.05 | — |
| | extract | | 25% | 16% | 18% | |
| | product | | | | | |
| 3. | Carrageenan and 500 mg/kg | 1.22 ± 0.13 | 1.36 ± 0.16 | 1.26 ± 0.05 | 1.16 ± 0.05 | — |
| | extract | | 11% | 3% | 0% | |
| | product twice (1 h | | | | | |
| | before and 1 h after | | | | | |
| | Carrageenan | | | | | |
| | administration) | | | | | |
| 4. | Carrageenan and | 1.3 ± 0.1 | 1.68 ± 0.16 | 1.4 ± 0.14 | 1.42 ± 0.08 | — |
| | 1000 mg/kg extract | | 29% | 8% | 9% | |
| | product | | | | | |
| 5. | Carrageenan and 15 mg/kg | 1.05 ± 0.1 | 1.51 ± 0.05 | 1.35 ± 0.08 | 1.18 ± 0.1 | — |
| | Indomethacin | | 44% | 29% | 12% | |
| | (positive control) | | | | | |

TABLE 2

| | | | Increase in rat hind paw volume after carrageenan | | | |
|---|---|---|---|---|---|---|
| | | Initial paw | administration as a function of time (h) | | | |
| Group | Treatment | volume (ml) | 3 h | 5 h | 24 h | 48 h |
| 1. | Carrageenan | 1.04 ± 0.13 | 2.08 ± 0.26 | 1.65 ± 0.20 | 1.54 ± 0.13 | 1.24 ± 0.05 |
| | (negative control) | | 100% | 59% | 48% | 19% |
| 2. | Carrageenan and 50 mg/kg | 1.07 ± 0.29 | 1.74 ± 0.22 | 1.42 ± 0.26 | 1.22 ± 0.16 | — |
| | extract product | | 63% | 33% | 14% | |
| 3. | Carrageenan and 50 mg/kg | 1.0 ± 0.08 | 1.2 ± 0.14 | 1.33 ± 0.21 | 1.08 ± 0.1 | — |
| | extract product | | 20% | 33% | 8% | |
| | twice (1 h before and | | | | | |
| | 1 h after carrageenan | | | | | |
| | administration) | | | | | |
| 4. | Carrageenan and 100 mg/kg | 0.97 ± 0.05 | 1.5 ± 0.05 | 1.23 ± 0.07 | 1.05 ± 0.08 | — |
| | extract product | | 55% | 27% | 8% | |
| | (1 h before | | | | | |
| | carrageenan | | | | | |
| | administration) | | | | | |
| 5. | Carrageenan and 100 mg/kg | 0.92 ± 0.08 | 1.55 ± 0.19 | 1.42 ± 0.8 | 1.20 ± 0.25 | — |
| | extract product | | 69% | 54% | 30% | |
| 6. | Carrageenan and 15 mg/kg | 1.05 ± 0.1 | 1.51 ± 0.05 | 1.35 ± 0.08 | 1.18 ± 0.1 | — |
| | Indomethacin | | 44% | 29% | 12% | |
| | (positive control) | | | | | |

### Example 5

### Demonstration of Anti-Inflammatory Activity of A. Melegueta Extract Product in Writhing Assay

The anti-inflammatory extract product of Example 1 was tested in vivo in mice using an acetic acid-induced writhing assay to evaluate the in vivo anti-inflammatory activity of the Aframomum melegueta extract products relative to a positive control. This writhing assay has been previously described [Inoue et al., Arzneimittelforschung, 41(3):235-239 (1991).]

The anti-inflammatory extract product of Example 1 was administered orally at two different doses (1000 mg/kg and 500 mg/kg) to two different groups of three mice one hour before intraperitoneal injection of the mice with acetic acid (0.5 vol. %, 20 ml/kg). An aqueous solution comprising 2 vol. % Tween 80 was administered as a negative control to another group of three mice. Aspirin was administered at 100 mg/kg as a positive control to a fourth group of three mice.

The number of writhings per animal group was observed during the period beginning at about five minutes after acetic acid challenge and ending at about ten minutes after acetic acid challenge. The average number of writhings per animal group is an indication of pain, e.g., inflammation-induced pain, and therefore a reduction in the number of writhings per unit time per animal group corresponds to a reduction in inflammation. The reduction in number of writhings can be expressed relative to the negative control value as a percentage of inflammation inhibition.

The average number of writhings per animal group receiving the vehicle control was 15. The average number of writhings per animal group receiving 1000 mg/kg anti-inflammatory extract product was 10, and thus the percentage of inflammation inhibition for this group was 33%. The average number of writhings per animal group receiving 500 mg/kg anti-inflammatory extract product was 12, and thus the percentage of inflammation inhibition for this group was 20%. The average number of writhings per animal group receiving 100 mg/kg aspirin was 3, and thus the percentage of inflammation inhibition for this group was 80%.

This example also demonstrates that Aframomum melegueta extract products can be used to reduce inflammation in an individual.

### Example 6

### Methods for Purifying an A. Melegueta Extract Product

This example describes several exemplary methods for purifying an Aframomum melegueta extract product. This example also provides data concerning the activity of individual compounds isolated from Aframomum melegueta extract products.

LC-MS Gradient (Method A):

Substances were separated on a Phenomenex® Luna C-8 reverse phase column, size 150×2 mm, particle size 3 μm, pore size 100 Å, equipped with a Phenomenex® SecurityGuard™ pre-column. The mobile phase consisted of 2 components: solvent A (0.5 vol. % ACS grade acetic acid in double-distilled de-ionized water, pH 3-3.5) and solvent B (100 vol. % acetonitrile). The mobile phase flow was adjusted to 0.25 ml/min, and generally a gradient mode was used as follows: 0-35 min: 95 vol. % solvent A-5 vol. % solvent A; 35-40 min: 5 vol. % solvent A; 40-45 min: 5 vol. % solvent A-95 vol. % solvent A (the balance of the mobile phase was solvent B).

HPLC Fractionation Procedure (Method B):

Compounds were separated on a Waters Symmetry Prep® RP 7 column, size 300×19 mm, particle size 7 μm. The mobile phase consisted of 2 components: solvent A (0.5 vol. % ACS grade acetic acid in double-distilled de-ionized water, pH 3-3.5), and solvent B (100 vol. % acetonitrile). The mobile phase flow was adjusted to 8 ml/min, and generally a gradient mode was used as follows: 0-35 min: 95 vol. % solvent A-5 vol. % solvent A; 35-40 min: 5 vol. % solvent A; 40-50 min 5 vol. % solvent A-95 vol. % solvent A (the balance of the mobile phase was solvent B).

The most prominent peaks were collected and their COX-2 inhibitory activity was evaluated using the in vitro COX-2 inhibition assay of Example 2. Additionally, these fractions were structurally analyzed by LC-MS. The LC-MS data demonstrated that members of the arylheptanoid family such as gingerols, shogaols and paradols are present in the anti-inflammatory extract product (along with other arylheptanoids).

Based on the generated analytical and COX-2 inhibitory data, paradols have the highest inhibitory activity in the COX-2 in vitro assay, followed by shogaols and gingerols. The COX-2 inhibitory activity for the paradols fraction (1 mg/mL) was 89%. The COX-2 inhibitory activity for the shogaols fraction (1 mg/mL) was 82%, whereas the COX-2 inhibitory activity for the control compound Vioxx® (1 mg/mL) was 88%. The COX-2 inhibitory activity for the gingerols fraction (1 mg/mL) was 30%.

Because the paradol fraction was the most active, it was subjected to further fractionation, and the fractions obtained were tested for COX-2 inhibitory activity as described below.

HPLC Subfractionation Procedure (Method C):

The paradol fraction obtained using Method B was further separated on a Phenomenex®. Curosil PFP column, size 250×4.60 mm, particle size 5 μm. The mobile phase consisted of 2 components: solvent A (double-distilled de-ionized water), and solvent B (100 vol. % acetonitrile). The mobile phase flow was adjusted to 0.5 ml/min, and generally an isocratic mode was used for all analyses as follows: 0-50 min: 40 vol. % solvent A-60 vol. % solvent B.

Subfractionation of the paradol fraction resulted in four major peaks. The COX-2 inhibitory activity of each peak was tested in the COX-2 in vitro assay of Example 2 at a concentration of 1 mg/mL. The second peak demonstrated the highest inhibitory activity (83%) followed by the first peak (74%), the fourth peak (14%), and the third peak (1%).

LC-MS structural analyses of these four peaks established that peaks 1 and 2 have an identical fragmentation pattern that corresponds to [6]-paradol. Peaks 3 and 4 are complex mixtures and were not fully resolved.

The invention is not limited to the embodiments described and exemplified above, but rather is capable of variation and modification without departure from the scope of the appended claims.

1. An anti-inflammatory composition comprising:
an anti-inflammatory extract product of a plant material of Aframomum melegueta; and,
at least one formulation agent selected from the group consisting of diluents, fillers, salts, binders and biologically acceptable carriers.

2. The anti-inflammatory composition of claim 1, wherein the plant material is selected from the group consisting of leaves, stems, flowers, fruits, seeds and roots.

3. The anti-inflammatory composition of claim 1, wherein the anti-inflammatory extract product comprises at least one compound in accordance with the following formula (I): 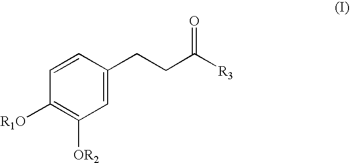
wherein R₁ is hydrogen or a C₁-C₄ moiety;
R₂ is hydrogen or a C₁-C₄ moiety; and,
R₃ is a C₁-C₁₀ moiety.

4. The anti-inflammatory composition of claim 3, wherein the anti-inflammatory extract product comprises at least one compound in accordance with the following formula (II):

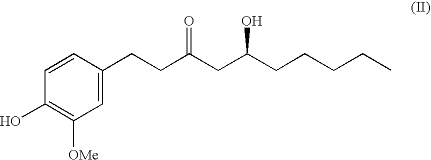

5. The anti-inflammatory composition of claim 3, wherein the anti-inflammatory extract product comprises at least one compound in accordance with the following formula (III):

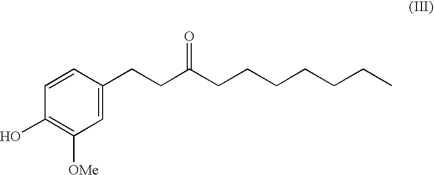

6. The anti-inflammatory composition of claim 3, wherein the anti-inflammatory extract product comprises at least one compound in accordance with the following formula (IV):

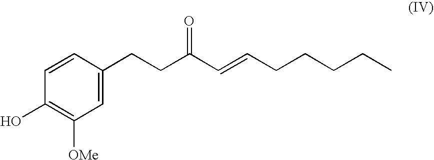

7. The anti-inflammatory composition of claim 1, wherein the anti-inflammatory extract product inhibits COX-2 activity.

8. The anti-inflammatory composition of claim 1, wherein the anti-inflammatory composition is formulated as a composition for topical administration.

9. The anti-inflammatory composition of claim 8, wherein the composition for topical administration is selected from the group consisting of gels, ointments, lotions, creams, sprays, drops, suppositories and transdermal patches.

10. The anti-inflammatory composition of claim 1, wherein the anti-inflammatory composition is formulated as a composition for oral administration.

11. The anti-inflammatory composition of claim 10, wherein the composition for oral administration is selected from the group consisting of pills, capsules and tablets.

12. A method of making an anti-inflammatory composition, comprising:
providing a plant material of an Aframomum melegueta plant;
extracting the plant material with a fluid;
collecting the fluid, thereby obtaining an anti-inflammatory extract product of the Aframomum melegueta plant; and,
formulating the anti-inflammatory extract product with at least one formulation agent selected from the group consisting of diluents, fillers, salts, binders and biologically acceptable carriers.

13. The method of claim 12, wherein the anti-inflammatory extract product is formulated as a nutraceutical composition.

14. The method of claim 12, wherein the anti-inflammatory extract product is formulated as a cosmeceutical composition.

15. The method of claim 12, wherein the anti-inflammatory extract product is formulated as a pharmaceutical composition.

16. The method of claim 12, further comprising macerating the plant material.

17. The method of claim 12, wherein the plant material is selected from the group consisting of leaves, stems, flowers, fruits, seeds and roots.

18. The method of claim 12, wherein the anti-inflammatory extract product comprises at least one compound in accordance with the following formula (I): 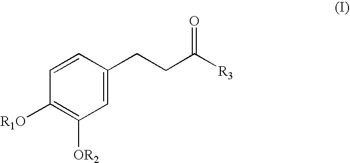
wherein R₁ is hydrogen or a C₁-C₄ moiety;
R₂ is hydrogen or a C₁-C₄ moiety; and,
R₃ is a C₁-C₁₀ moiety.

19. A method of treating or preventing an inflammatory response, comprising:
administering a therapeutically effective amount of a composition comprising an anti-inflammatory extract product of a plant material of an Aframomum melegueta plant to an individual.

20. The method of claim 19, wherein the plant material is selected from the group consisting of leaves, stems, flowers, fruits and roots.

21. The method of claim 19, wherein the anti-inflammatory extract product comprises at least one compound in accordance with the following formula (I): 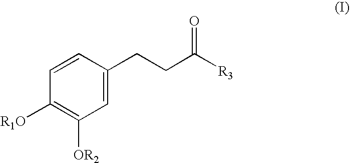
wherein R₁ is hydrogen or a C₁-C₄ moiety;
R₂ is hydrogen or a C₁-C₄ moiety; and,
R₃ is a C₁-C₁₀ moiety.

22. The method of claim 22, wherein the individual is a mammal.

23. The method of claim 22, wherein the inflammatory response is associated with a condition selected from the group consisting of autoimmune diseases, arthritic diseases, dermatitis, and allergic diseases.

24. A method of treating or preventing cancer, comprising:
administering a therapeutically effective amount of a composition comprising an extract product of a plant material of an Aframomum melegueta plant to an individual.

25. An article of manufacture comprising a composition comprising an extract product of a plant material of an Aframomum melegueta plant and a set of instructions for administering the composition to treat or prevent an indication selected from inflammation and cancer.
